(19) United States
(12) Patent Application Publication
(10) Pub. No.: US 2021/0198161 A1
(43) Pub. Date: Jul. 1, 2021

(54) AN APPARATUS AND A METHOD FOR COMPOSTING ORGANIC MATERIAL

(71) Applicant: BIOPALLO SYSTEMS OY, Kuopio (FI)

(72) Inventor: Anssi SUHONEN, Kuopio (FI)

(21) Appl. No.: 17/270,287

(22) PCT Filed: Jul. 11, 2019
(86) PCT No.: PCT/FI2019/050543

§ 371 Date: Feb. 22, 2021

(30) Foreign Application Priority Data
Aug. 24, 2018 (FI) 20185697

(51) Int. Cl. C05F 17/943; C05F 17/971

(52) U.S. Cl. CPC C05F 17/943; C05F 17/971

(57) ABSTRACT

An apparatus and a method for composting organic material. The apparatus comprises a container (10) having a first end (11), a second end (12) in the form of a hemispherical wall, and a jacket (13) extending between said ends. The container operates in an inclined (al) position. A transporting and mixing device (30) in the container transports organic material in a transport direction (L1) from the first to the second end and simultaneously mixes the organic material. The transporting and mixing device directs the organic material towards the hemispheric wall, whereby the organic material is first directed upwards along the hemispheric wall and then turned back against the transport direction. A fluffing device (40) in the container comprises a shaft (41) being perpendicular to the transport direction (L1) and cutter blades (50) extending outwards from the shaft (41).

### FIELD

The invention relates to an apparatus and a method for composting organic material.

The apparatus for composting organic material may include a container with at least one inlet opening for the organic material and at least one outlet opening for the composted material and a transporting and mixing device within the container for transporting and mixing the material inside the container.

Material for regulating the oxygen content and/or the bacteria and/or the enzymes for agitation of the composting process may be added to the container. The organic material will compost in the container. The composted material may then be removed from the at least one outlet opening to a site outside the container in order to be stabilized.

### BACKGROUND

There are various prior art methods for composting organic material, such as biowaste and biomass and various devices used in the methods. Such devices are, for example, tunnel composters, drum composters and silo composters. The material to be composted in these composters is fed to the composter after which the conditions in the composters are regulated so that the composting process begins and proceeds in a functional way. Air may be fed to the composter during the composting process in order to supply oxygen into the composting process.

The prior art composting methods require a long time, e.g. usually several weeks, for the organic material to compost and the control of the composting process is difficult in the prior art methods. Another problem in prior art methods is that the organic material composts heterogeneously. It is also difficult to verify the proceeding of the stages of the process and the hygiene of the process in prior art composting methods. The control of the odour is also not efficient in current composting methods. The workers may even be exposed to health hazards in prior art composters.

DE 3844700 discloses a composting device comprising a container with a lower portion and an upper portion. The organic material is introduced from the side to the lower portion of the container to a cutting and mixing device. The material is further transported upwards with a screw. The outlet opening is at the upper end of the upper portion of the container.

EP 1 387 817 discloses a method and an apparatus for composting organic material. The apparatus comprises a container having a first end and a second end formed as a hemispherical wall. A transport and mixing device transports organic material from the first end to the second end of the container.

### SUMMARY

An object of the present invention is to present an improved apparatus and method for composting organic material.

The apparatus for composting organic material according to the invention is defined in claim 1.

The method for composting organic material according to the invention is defined in claim 9.

The apparatus for composting organic material comprises

a container having a first end, a second opposite end, and a jacket extending between the first end and the second end, the second end being formed as a hemispherical wall, the container comprising at least one inlet opening for the organic material and at least one outlet opening for composted material, the container operating in an inclined position in which the second end of the container is on a higher horizontal level than the first end of the container,

a transporting and mixing device positioned in the container for transporting organic material in a transport direction from the first end to the second end of the container and simultaneously mixing the organic material, the transporting and mixing device directing the organic material towards the hemispheric wall, whereby the organic material is first directed upwards along the hemispheric wall and then turned back against the transport direction.

The apparatus is characterized in that

a fluffing device is positioned in the container for cutting the organic material into a smaller particle size and for throwing at least a portion of the organic material backwards towards the first end of the container, the fluffing device comprising a shaft extending substantially perpendicularly in relation to the transport direction and cutter blades extending substantially perpendicularly outwards from the shaft.

The method for composting organic material comprises

providing organic material from at least one inlet opening to a container having a first end and an opposite second end formed as a hemispheric wall, the container operating in an inclined position in which the second end of the container is on a higher horizontal level than a first end of the container,

providing additional material for regulating the oxygen content and/or bacteria and/or enzymes to the container in order to agitate the composting process in the container,

transporting the organic material with a transporting and mixing device in a transport direction from the first end towards the hemispheric wall at the second end of the container, whereby the organic material is first directed upwards along the hemispheric wall and then turned back towards the first end of the container, the organic material also being mixed during the transport,

removing the composted material from at least one outlet opening in the container after the composting process within the container has been completed,

The method is characterised in

cutting the organic material into a smaller particle size and throwing at least a portion of the organic material backwards towards the first end of the container with a fluffing device comprising a shaft extending substantially perpendicularly in relation to the transport direction and cutter blades extending outwards from the shaft.

The apparatus for composting organic material provides a fast, homogeneous and flexible composting process within the container. It is easy to provide suitable measurements within the container in order to be able to verify how the composting process proceeds. The apparatus for composting organic material is also simple and reliable.

The time to compost a batch of organic material in the apparatus for composting organic material is only 1 to 3 days. The composted material may then be removed from the at least one outlet opening to a site outside the container in order to be stabilized.

The fluffing device will intensify the cutting and/or chopping of the organic material into a smaller particle size within the container. The smaller particle size will contribute to a more efficient oxidation of the organic material within the container.

The fluffing device will also throw at least a portion of the organic material backwards towards the first end of the container in order to achieve an efficient rotation and mixing of the organic material within the container.

The fluffing device will also contribute to eliminate the vaulting of the organic material on the walls of the container at the second end of the container. This vaulting may be a problem especially when the moisture content of the organic material is high within the container.

At least the moisture content and the temperature within the container may advantageously be measured by a suitable moisture sensor and temperature sensor positioned within the container. The moisture content within the container may be regulated by providing fresh air to the container. The moisture content of the air that is supplied into the container may be regulated, whereby also the moisture content within the container becomes adjusted. The supply of fresh air into the container will naturally also provide oxygen into the container.

The pH-value of the organic material within the container may further be measured. Acidity regulating material may then be added to the container in order to adjust the acidity of the organic material within the container. The acidity of the organic material has an influence on the functioning of the bacteria de-composting the organic material within the container. By adding lime or some other acidity controlling material into the container, it is possible to keep the pH-value of the organic material within the container at a suitable level for an efficient composting process.

The optimal mixing and/or oxidation of the organic material within the container may be regulated by regulating the inclination angle of the container and/or by regulating the rotation speed of the screws in the transporting and mixing device and/or by regulating the rotation speed of the fluffing device. The air circulation within the container and/or the removal of moisture from the container may further be regulated by regulating the supply of air to the container.

### DRAWINGS

The invention will in the following be described in greater detail by means of preferred embodiments with reference to the attached drawings, in which:

FIG. 1 shows a side view of an apparatus for composting organic material,

FIG. 2 shows a side view of the transporting and mixing device,

FIG. 3 shows an upper view of the transporting and mixing device and the fluffing device,

FIG. 4 shows an enlarged upper view of the transporting and mixing device and the fluffing device,

FIG. 5 shows an axonometric view of the transporting and mixing device and the fluffing device,

FIG. 6 shows an axonometric view of the fluffing device,

FIG. 7 shows an axonometric view of a blade in the fluffing device.

### DETAILED DESCRIPTION

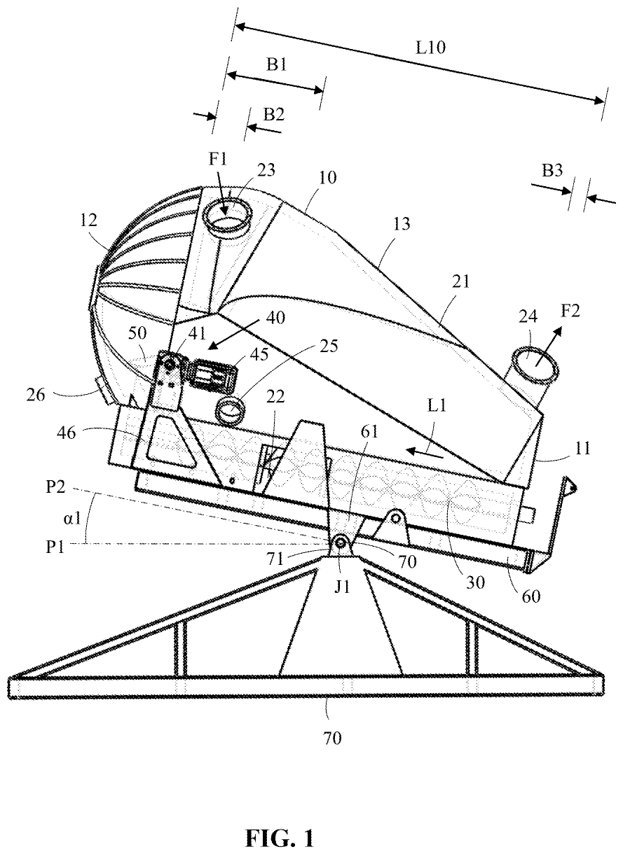

FIG. 1 shows a side view of an apparatus for composting organic material.

The apparatus for composting organic material comprises a container 10, a transporting and mixing device 30, and a fluffing device 40.

The container 10, the transporting and mixing device 30, and a fluffing device 40 are supported on a first frame structure 60. The first frame structure 60 is positioned below the transporting and mixing device 30.

The container 10 has a first end 11, a second opposite end 12, and a jacket 13 extending between the first end 11 and the second end 12. The second end 12 of the container 10 may be formed as a hemispherical wall. The jacket 13 of the container 10 may have a substantially conical form so that the cross section of the jacket 13 contracts from the beginning of the hemispherical wall at the second end 12 of the container 10 towards the first end 11 of the container 10. The first end 11 of the container may be inclined.

The container 10 in this embodiment has at least one inlet opening 21 for the organic material and at least one outlet opening 22 for composted material. The container 10 in this embodiment has further at least a first air opening 23 through which air can be directed into the interior of the container 10 and at least a second air opening 24 through which air can be directed out from the interior of the container 10. Fresh air and thereby oxygen can be provided to the interior of the container 10 from the first air opening 23. The supply of oxygen to the container 10 will contribute to the speed and to the control of the composting process. The container 10 in this embodiment has further measuring pockets 25, 26 for measuring the temperature within the container 10.

The container 10 may in an embodiment be provided with one first air opening 23 and one second air opening 24.

The container 10 may be operated in an inclined al position in which the second end 12 of the container 10 may be on a higher horizontal level than the first end 11 of the container 10.

The transporting and mixing device 30 is positioned in the container 10. The transporting and mixing device 30 may be positioned substantially at a bottom of the container 10. The transporting and mixing device 30 may transport organic material in a transport direction L1 from the first end 11 to the second end 12 of the container 10. The transporting and mixing device 30 may mix the organic material simultaneously during the transport of the organic material from the first end 11 to the second end 12 of the container 10. The transporting and mixing device 30 may direct the organic material towards the hemispheric wall at the second end 12 of the container 10. The organic material is first directed upwards along the hemispheric wall at the second end 12 of the container 10. The organic material is after that turned back against the transport direction L1 of the organic material in the upper portion of the hemispherical wall. The organic material will fall down from the upper portion of the hemispherical wall and the upper portion of the jacket 13 of the container 10 on the transporting and mixing device 30 in order to be transported again in the transport direction L1 against the second end 12 of the container 10.

The fluffing device 40 is positioned in the container 10. The fluffing device 40 may comprise a shaft 41 extending substantially perpendicularly in relation to the transport direction L1. The shaft 41 may be provided with cutter blades 50 extending outwards from the shaft 41. The cutter blades 50 may extend perpendicularly outwards from the shaft 41. The cutter blades 50 may be attached to the shaft 41 so that rotation of the shaft 41 will also rotate the cutter blades 50. The fluffing device 40 may be driven by an electric motor 45. The electric motor 45 may be connected to the shaft 41 of the fluffing device 40. The fluffing device 40 is used to cut and fluff the organic material. The shaft 41 of the fluffing device 40 may be positioned substantially at the beginning of the spherical wall at the second end 12 of the container 10.

The position of the shaft 41 of the fluffing device 40 shown in the figure i.e. substantially at the beginning of the spherical wall at the second end 12 of the container 10 is an advantageous position. The fluffing device 40 may in this position cut and fluff the organic material just before it is entering the hemispheric wall at the second end 12 of the container 10. The cutter blades 50 of the fluffing device 40 may be formed so that a portion of the organic material is thrown back towards the first end 11 of the container 10 by the cutter blades 50 when they rotate with the shaft 41 of the fluffing device 40.

The beginning of the hemispherical wall is the junction between the jacket 13 and the hemispherical wall at the second end 12 of the container 10. The beginning of the hemispheric wall is thus the point at which a diameter of a cross section of the hemispherical wall reaches the greatest value.

The shaft 41 of the fluffing device 40 may be positioned at a first distance B1 from the beginning of the spherical wall at the second 12 of the container 10. This first distance B1 may be in the range of 0-20%, preferably in the range of 0-10% of a first length L10 extending from the first end 11 of the container 10 to the beginning of the hemispheric wall at the send end 12 of the container 10. The first distance B1 and the first length L10 may extend in the transport direction L1.

The first air opening 23 may be positioned in an upper portion of the container 10. The first air opening 23 may be positioned substantially above the fluffing device 40. The first air opening 23 may be positioned at a second distance B2 from the beginning of the spherical wall at the second end 12 of the container 10. This second distance B2 may be in the range of 0-20%, preferably in the range of 0-10% of the first length L10 extending from the first end 11 of the container 10 to the beginning of the hemispheric wall at the send end 12 of the container 10. The second distance B2 may also extend in the transport direction L1.

A flow of fresh inlet air F1 may thus be directed towards the flow of organic material thrown backwards in the container with the fluffing device 40. The rotational speed of the fluffing device 40 may be regulated so that the portion of organic material which is thrown backwards towards the first end 11 of the container 10 with the fluffing device 40 is transferred into a mist containing droplets of organic material. The fresh inlet air F1 blown into the container 10 will thus be directed towards the mist of organic material, whereby an efficient oxidation of the organic material is achieved.

The second air opening 24 may be positioned in an upper portion of the container 10. The second air opening 24 may be positioned above the transporting and mixing device 30. The second air opening 24 may be positioned at a third distance B3 from the first end 11 of the container 10. This third distance B3 may be in the range of 0-20%, preferably in the range of 0-10% of the first length L10 extending from the first end 11 of the container 10 to the beginning of the hemispherical wall at the second end 12 of the container 10. The third distance B3 may also extend in the transport direction L1.

A continuous air circulation may be provided through the container 10 so that air F1 is directed into the container 10 from the first air opening 23 and air F2 is directed out from the container 10 from the second air opening 24. A blower positioned outside the container 10 may be used to produce the air circulation through the container 10.

The first air opening 23 and the second air opening 24 may be positioned in an upper portion of the jacket 13 of the container 10. The first air opening 23 and the second air opening 24 may thus be positioned above the transporting and mixing device 40. The first air opening 23 may further be positioned substantially above the fluffing device 40.

The container 10 may be adjustable between a horizontal first position P1 and an inclined al second position P2 in which the second end 12 of the container 10 is on a higher level than the first end 11 of the container 10. The adjustability of the container 10 between the first and the second position P1, P2 may be achieved by supporting the container 10 pivotably J1 on a second frame structure 70. The container 10 may be arranged to be turnable around the pivot point J1 between the horizontal first position P1 and the inclined al second position P2. The pivot J1 support may be arranged between the first frame structure 60 and the second frame structure 70. The first frame structure 60 may comprise downwardly directed first lugs 61 at each side of the first frame structure 60. The second frame structure 70 may comprise corresponding upwardly directed second lugs 71 at each side of the second frame structure 70. The first and the second lugs 61, 71 may comprise a hole so that a shaft 80 may pass through the holes in the first and the second lugs 61, 71. The first frame structure 60 and thereby also the container 10 is thus pivotable around the shaft 80.

The turning of the container 10 between the first position P1 and the second position P2 and vice a versa may be done with any suitable actuators e.g. with hydraulic cylinders or electric motors connected to cogwheels. (This is not for clarity reasons shown in the figures).

The container 10 may be operated in batch runs. The container 10 may first be positioned in the horizontal first position P1 and filled with organic material through the inlet opening 21 to approximately one third of the volume of the container 10. The container 10 may thereafter be turned into the inclined second position P2 in which second position P2 the container 10 is operated during the composting process. The transporting and mixing device 30 may then be operated in desired intervals during the composting process. The fluffing device 40 may also be operated in desired intervals during the composting process. The composting conditions within the container 10 may be measured continuously or at certain intervals during the composting process. The transporting and mixing device 30 as well as the fluffing device 40 may be operated based on the composting conditions within the container 10 determined from the results of the measurements.

The transporting and mixing device 30 and the fluffing device 40 may each have a driving motor of their own. This would mean that the two devices 30, 40 may be operated so that the operation intervals of the two devices 30, 40 coincide fully or partly or so that there is no overlapping of the operation intervals of the two devices 30, 40. Also the rotational speed of each device 30, 40 could then be regulated independently. The transporting and mixing device 30 and the fluffing device 40 may on the other hand be operated by a common driving motor. One of the two devices 30, 40 could be connected directly to the driving motor and the other device 30, 40 could be connected through a transmission to the driving motor or both devices 30, 40 could be connected through a transmission to the driving motor. This would mean that the two devices 30, 40 may be operated only in synchronism with each other. The rotational speed of the devices 30, 40 could be different, but the ratio between the rotational speeds of the devices 30, 40 would be constant. This ratio could be determined by the transmission.

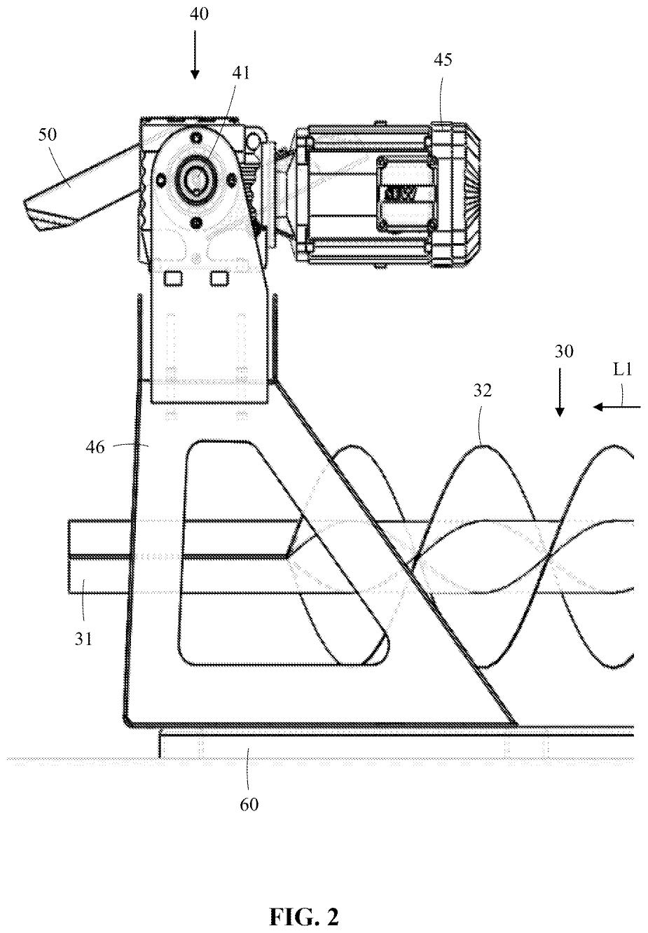

FIG. 2 shows a side view of the transporting and mixing device.

The transporting and mixing device 30 may comprise a cylindrical shaft 31 provided with a screw 32 extending outwards from the outer perimeter of the shaft 31. Rotation of the shaft 31 rotates the screw 32 whereby material is transported with the screw 32 from the first end 11 of the container 10 to the second end 12 of the container 10. The transporting and mixing device 30 may be driven by an electric motor (not shown in the figure) connected to the shaft 31 of the transporting and mixing device 30. The threading of the screw 32 transports the material in the axial direction of the transporting and mixing device 30 when the shaft 31 is rotated.

The fluffing device 40 may comprise a shaft 41 extending in a direction which is substantially perpendicular in relation to the transport direction L1. The shaft 41 may be positioned within the container 10 substantially at the beginning of the spherical wall at the second end 12 of the container 10. The fluffing device 40 may further comprise cutter blades 50 extending perpendicularly outwards from the shaft 41.

The fluffing device 40 may be driven by an electric motor 45 connected to the shaft 41 of the fluffing device 40.

Both ends of the shaft 41 of the fluffing device 40 may be rotatably supported e.g. through bearings on a support structure 46. The support structure 46 may be supported on the first frame construction 50. The fluffing device 40 may be positioned above the transporting and mixing device 30 in the vertical direction. The cutter blades 50 of the fluffing device 40 may thus rotate freely without interfering with the screw 32 of the transporting and mixing device 30.

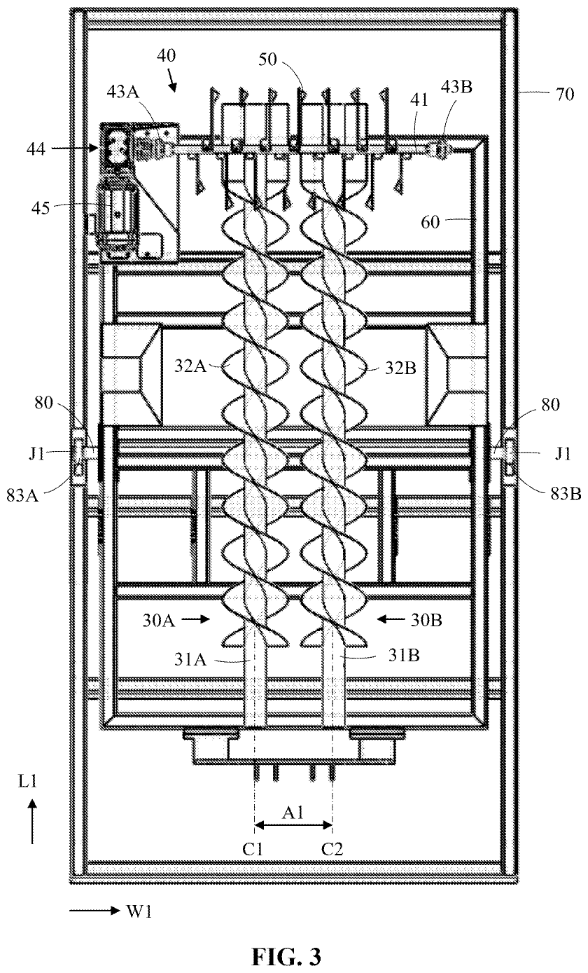

FIG. 3 shows an upper view of the mixing and transport device and the fluffing device.

This transporting and mixing device 30 in this embodiment comprises two longitudinal in the length direction L1 of the apparatus directed screw means 30A, 30B. The length direction L1 of the apparatus coincides with the transport direction L1 of the apparatus. Each screw means 30A, 30B comprises a shaft 31A, 31B and a screw 32A, 32B extending outwards from the perimeter of the shaft 31A, 31B. The longitudinal centre axes C1, C2 of the shafts 31A, 31B of the two screw means 30A, 30B are parallel and positioned at a distance A1 from each other. The tips of the screws 32A, 32B of the screw means 30A, 30B are positioned at a distance from each other so that the screws 32A, 32B can rotated without contacting each other and without disturbing each other.

The screws 32A, 32B are open i.e. there is no pipe surrounding the screws 32A, 32B. The threads of the screws 32A, 32B in both screw means 30A, 30B are directed so that the material is transported with the screw 32A, 32B from the first end 11 to the second end 12 of the container 10. The threads of the screws 32A, 32B may be identical.

The shafts 31A, 31B of the screw means 30A, 30B may be supported via bearings to the first frame construction 60 (this is not for clarity reasons shown in the figures).

One end of the shafts 31A, 31B of the screw means 30A, 30B may be connected to a drive motor (this is not for clarity reasons shown in the figures). The two shafts 31A, 31B may be driven by a common drive motor connected through a transmission to the two shafts 31A, 31B. The other possibility is that each of the shafts 31A, 31B is driven by a drive motor of their own. The drive motor may be an electric motor.

Each end of the shaft 41 of the fluffing device 40 is rotatably supported via a bearing 43A, 43B on the first frame structure 60. A first end of the shaft 41 of the fluffing device 40 may be connected via a transmission 44 to the drive motor 45. The rotational speed of the shaft 41 of the fluffing device 40 may be the same or it may be different i.e. greater or smaller than the rotational speed of the shaft of the drive motor 45. The ratio between the rotational speeds of the fluffing device 40 and the drive motor 45 may be determined by the transmission.

The first frame structure 60 may be supported via a shaft 80 on the second frame construction 70. The shaft 80 may extend in the width direction W1 of the apparatus over the whole width of the first frame structure 60 or the shaft 80 may be formed of two pins extending outwards from the sides of the first frame construction 60. The end of the shaft 80 may be supported with bearings 83A, 83B on the second frame construction 70. The shaft 80 forms a pivot point J1 between the first frame construction 60 and the second frame construction 70. The first frame construction 60 may be turned in relation to the second frame construction 70 around the pivot point J1.

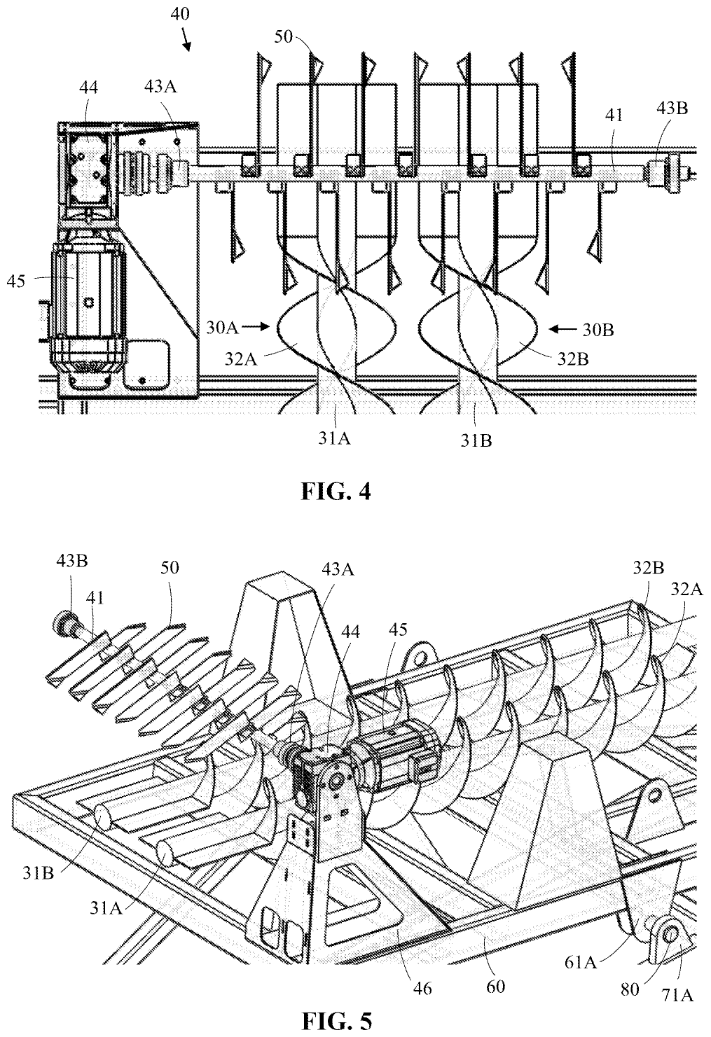

FIG. 4 shows an enlarged upper view of the transporting and mixing device and the fluffing device and FIG. 5 shows an axonometric view of the transporting and mixing device and the fluffing device.

The fluffing device 40 is positioned above the transporting and mixing device 30A, 30B so that the cutter blades 50 of the fluffing device 40 may rotate freely without contacting the screws 32A, 32B of the screw means 30A, 30B.

The drive motor 45 and the transmission 44 is supported via a support structure 46 on the first frame structure 60.

One end of the shaft 41 of the fluffing device 40 is connected to the transmission 44 of the drive motor 45.

The ends of the shaft 41 of the fluffing device 40 may be supported via bearings 43A, 43B on the first frame structure 60. This support is not for clarity reasons shown in the figures.

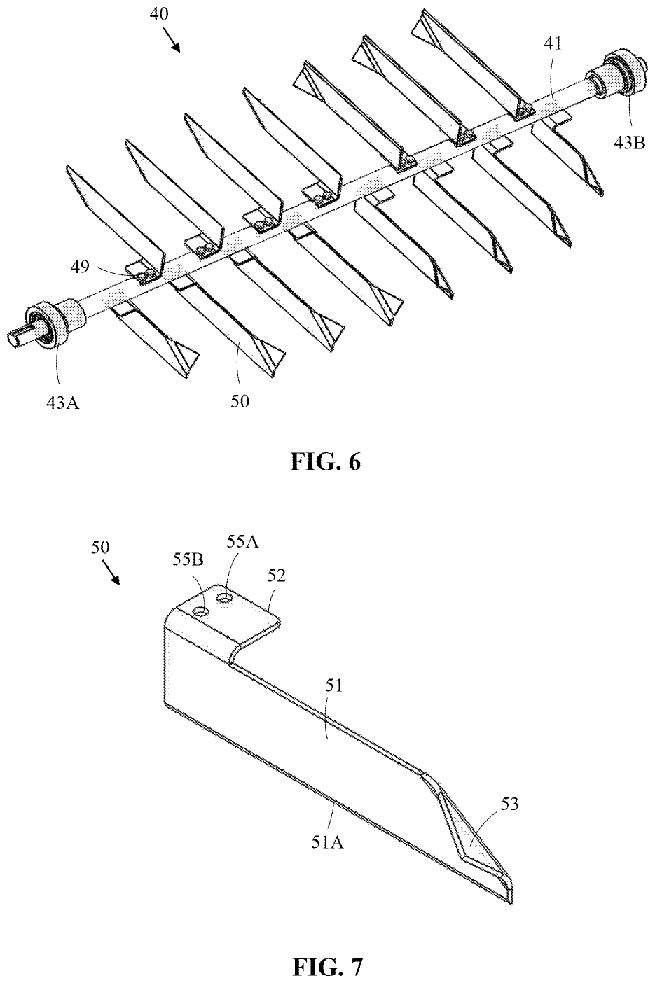

FIG. 6 shows an axonometric view of the fluffing device and FIG. 7 shows an axonometric view of a blade in the fluffing device.

The fluffing device 40 comprises a shaft 41 which may be supported via bearings 43A, 43B on the first frame structure 60. Cutter blades 50 may be attached to the shaft 41 so that the cutter blades 42 protrude radially outwards from the shaft 41. The cutter blades 50 may be attached with bolts 49 to the shaft 41 of the fluffing device 40. The shaft 41 of the fluffing device 40 may be provided with threaded openings receiving the thread of the bolts 49. The other possibility is that the shaft 41 of the fluffing device 40 is provided with holes passing through the shaft 41. The bolts 49 are in such case tightened with nuts threaded on the end of the bolts 49 on the opposite side of the shaft 41 of the fluffing device 40.

The cutter blades 50 may comprise a main blade portion 51 having a substantially rectangular form, a fastening portion 52 extending substantially perpendicularly from the main blade portion 51 at an inner end of the main blade portion 51, and an inclined portion 53 at an outer end of the main blade portion 51. The inclined portion 53 may be made by bending an upper corner of the main blade portion 51 at the outer end of the main blade portion 51. The lower edge 51A of the main blade portion 51 may be sharp in order to be able to effectively cut the material to be composted.

The function of the straight portion of the main portion 51 of the cutter blade 50 is to cut the material to be composted.

The function of the inclined portion 53 of the main portion 51 of the cutter blade 50 is to throw a portion of the material to be composted backwards against the transport direction L1.

The driving motor 45 and the transmission 44 driving the shaft 41 of the fluffing device 40 is positioned outside the container 10. At least the end of the shaft 41 of the fluffing device 40 to which the transmission 44 is connected passes thus trough an opening in the wall of the container 10 to the outside of the container 10. The shaft 41 of the fluffing device 40 may be sealed with a sealing in said opening of the container 10.

The figures show an embodiment in which the transporting and mixing device 30 is formed of two screw means 30A, 30B. This is an advantageous embodiment, but the invention is not restricted to this embodiment. The transporting and mixing device 30 could be formed of any number of screw means 30A, 30B. The transporting and mixing device 30 may thus comprise one screw means, two screw means, three screw means, four screw means etc. The transporting and mixing device 30 may on the other hand comprise at least one screw means, at least two screw means, at least three screw means, at least four screw means etc.

The volume of the container 10 may be adapted according to the need. The volume of the container 10 may e.g. be in the range of 1 to 100 m³. The volume of the container 10 may in an embodiment be in the range of 5 to 6 m³ and the weight of the material to be composted in one batch may be in the range of 1500 to 2000 kg. The container 10 may be filled with material to be composted approximately to one third of the volume of the container 10. There should be enough of air space within the container 10 in order for the composting process to proceed properly. This air space may be provided in the upper portion of the container 10 above the organic material.

The angle α of deflection of the container 10 may be in the range of 10 to 50 degrees, preferably substantially 30 degrees.

The figures show an embodiment in which fresh air F1 is directed into the container 10 from the first air opening 23 positioned in the vicinity of the beginning of the hemispherical wall at the second end 12 of the container 10 and air F2 is directed out from the container 10 from the second air opening 24 positioned in the vicinity of the first end 11 of the container 10. This is an advantageous solution as the fresh air will be blown directly against the portion of organic material that is thrown backwards by the fluffing device 40. It is, however, possible to change the flow direction of the air flow so that air is directed in from the second air opening 24 and out from the first air opening 23 in the container 10.

It will be obvious to a person skilled in the art that, as the technology advances, the inventive concept can be implemented in various ways. The invention and its embodiments are not limited to the examples described above but may vary within the scope of the claims.

1. An apparatus for composting organic material comprising a container having a first end, a second opposite end, and a jacket extending between the first end and the second end, the second end being formed as a hemispherical wall, the container comprising at least one inlet opening for the organic material and at least one outlet opening for composted material, the container operating in an inclined position in which the second end of the container is on a higher horizontal level than the first end of the container,
a transporting and mixing device positioned in the container for transporting organic material in a transport direction from the first end to the second end of the container and simultaneously mixing the organic material, the transporting and mixing device directing the organic material towards the hemispheric wall, whereby the organic material is first directed upwards along the hemispheric wall and then turned back against the transport direction,
wherein
a fluffing device is positioned in the container for cutting the organic material into a smaller particle size and for throwing at least a portion of the organic material backwards towards the first end of the container, the fluffing device comprising a shaft extending substantially perpendicularly in relation to the transport direction and cutter blades extending outwards from the shaft.

2. The apparatus according to claim 1, wherein the shaft of the fluffing device is positioned at a first distance from the beginning of the hemispherical wall at the second end of the container, said first distance being in the range of 0 to 20% of a first length extending from the beginning of the hemispherical wall at the second end of the container to the first end of the container.

3. The apparatus according to claim 1, wherein the shaft of the fluffing device is positioned substantially at a beginning of the hemispherical wall at the second end of the container.

4. The apparatus according to claim 1, wherein the cutter blades comprises an inclined portion at a radially outer end of the cutter blades, said inclined portion throwing at least a portion of the material to be composted backwards towards the first end of the container.

5. The apparatus according to claim 1, wherein the fluffing device is positioned above the transporting and mixing device.

6. The apparatus according to claim 1, wherein the transporting and mixing device is formed of at least two screw means, each of the screw means comprising a shaft and a screw protruding outwards from a perimeter of the shaft.

7. The apparatus according to claim 1, wherein the container is adjustable between a horizontal position and an inclined position in which the second end of the container is on a higher level than the first end of the container.

8. The apparatus according to claim 7, wherein the container is pivotably supported on a second frame structure so that the container is turnable around the pivot point between the horizontal position and the inclined position.

9. The apparatus according to claim 1, wherein the jacket of the container comprises a first air opening through which air can be directed into the interior of the container and a second air opening through which air can be directed out from the interior of the container.

10. The apparatus according to claim 9, wherein the first air opening is positioned substantially above the fluffing device.

11. The apparatus according to claim 9, wherein the first air opening is positioned at a second distance from the beginning of the hemispherical wall at the second end of the container, said second distance being in the range of 0 to 20% of a first length extending from the beginning of the hemispherical wall at the second end of the container to the first end of the container.

12. The apparatus according to claim 9, wherein the second air opening is positioned at a third distance from the first end of the container above the transporting and mixing device, said third distance being in the range of 0 to 20% of a first length extending from the beginning of the hemispherical wall at the second end of the container to the first end of the container.

13. A method for composting organic material comprising
providing organic material from at least one inlet opening to a container having a first end and an opposite second end formed as a hemispheric wall, the container operating in an inclined position in which the second end of the container is on a higher horizontal level than a first end of the container,
providing additional material for regulating the oxygen content and/or bacteria and/or enzymes to the container in order to agitate the composting process in the container,
transporting the organic material with a transporting and mixing device in a transport direction from the first end towards the hemispheric wall at the second end of the container, whereby the organic material is first directed upwards along the hemispheric wall and then turned back towards the first end of the container, the organic material also being mixed during the transport,
removing the composted material from at least one outlet opening in the container after the composting process within the container has been completed,
wherein
cutting the organic material into a smaller particle size and throwing at least a portion of the organic material backwards towards the first end of the container with a fluffing device comprising a shaft extending substantially perpendicularly in relation to the transport direction and cutter blades extending outwards from the shaft.
